(19) United States
(12) Patent Application Publication
(10) Pub. No.: US 2008/0145446 A1
(43) Pub. Date: Jun. 19, 2008

(54) Multi-purpose hand and/or skin cleaner, sanitizer, drug delivery and exercise system

(76) Inventors: Chris Johnson, Laguna Beach, CA (US); Drew O'Connell, Cold Spring Harbor, NY (US)

Correspondence Address: Melvin I. Stoltz, 51 Cherry Street, Milford, CT, 06460, US

(21) Appl. No.: 11/893,052

(22) Filed: Aug. 13, 2007

Related U.S. Application Data
(60) Provisional application No. 60/874,999, filed on Dec. 14, 2006.

(51) Int. Cl. A01N 59/26; A01N 37/00; A01P 1/00; A61K 31/05; A61K 31/164; A61K 31/215; A61K 31/60; A61K 33/42; A61Q 19/10; C11D 3/20

(52) U.S. Cl. 424/601; 510/130; 510/138; 514/165; 514/535; 514/625; 514/726

(57) ABSTRACT

By providing a reusable support base or carrier which is constructed for being easily held by the user in the person's hand, and incorporating additives, therapeutic agents and/or medicinal components therein, a highly effective and easily employed, multi-purpose hand and/or skin cleaner, sanitizer, drug delivery, and exercise system is realized. Preferably, the support base or carrier is formed from soft, pliable, compressible and/or flexible material which is easily manipulated by the user in the person's hand or on the skin surface of other areas of the person's body. By employing selected additives or combinations thereof, a multi-purpose, highly effective, and unique hand or skin cleaning, sanitizing, and drug delivery system is realized in a system which also improves and enhances the individual's ability to obtain exercising of the hand.

### RELATED DATA

This application is related to U.S. Provisional Patent Application Ser. No. 60/874,999, filed Dec. 14, 2006, entitled Multi-Purpose Hand and/or Skin Cleaner, Sanitizer, Drug Delivery, and Exercise System.

### TECHNICAL FIELD

This invention relates to hand and/or skin cleansers and sanitizers and, more particularly, to a self-contained, holdable and flexible carrier system incorporating additives therein.

### BACKGROUND ART

During the last several decades, there has been an ever increasing awareness of the impact micro organisms have on individuals and the overall health of individuals. In this regard, individuals have become aware that micro organisms produce a wide variety of sicknesses and ailments to which many individuals are exposed and are caused to suffer. In addition, it has been found that by employing enhanced sanitary practices, exposure to disease causing micro organisms can be eliminated or substantially reduced. In particular, it has been found that by paying careful attention to cleaning and/or sanitizing of one's hands, a substantial reduction of disease transmission can be realized.

In this regard, it has become well established that infectious diseases are commonly spread through hand-to-hand contact causing diseases such as the common cold, flu, gastrointestinal disorders, and infectious diarrhea to be transmitted. Furthermore, it has been found that hand hygiene also contributes to food-related illness, such as salmonella, and E. coli infection.

In order to address this ever-increasing problem, individuals are encouraged to wash their hands repeatedly throughout the day, using soap and water, or other sanitizing products. In this regard, in order to satisfy the needs and desires of individual for ever increasing protection against disease transmitting micro organisms, anti-microbial and/or anti-bacterial compositions have been developed for use by individuals. However, these prior art compositions typically require an individual to be at home or at work in order to use these products. In addition, most of these prior products also require the use of water for properly enabling the products to provide the desired result.

In spite of the substantial effort that has been expended in developing suitable compositions for meeting the sanitation needs of individuals, these prior art products have had only minimal effect on the continuing problem that exists. In particular, the inherent limitations caused by these products have prevented these products from obtaining wide acceptance and use.

In particular, most prior products require the use of water for being effectively employed. As a result, individuals are unable to employ the sanitizing products when exposure to micro organisms, bacteria, and viruses often occur, such as while shopping, walking, or during a normal daily activities outside of the home or office. In addition, the prior products have failed to address the substantial risk of exposure being suffered by children, who are continuously exposed to a wide variety of microorganisms, bacteria, and viruses in numerous daily activities, and who typically avoid the use of soap and water, as well as other cleansing compositions.

Consequently, it is a principal object of the present invention to provide a highly effective and easily employed, waterless, multi-purpose product which is capable of providing hand and/or skin cleaning and sanitizing in any location and at any time whenever desired.

Another object of the present invention is to provide a waterless, multi-purpose product having the characteristic features described above which is easily employed by merely rubbing or compressing the product in one's hand, thereby enabling the anti-microbial and anti-bacterial ingredients contained therein to be effectively released for disinfecting the hand and/or skin surface.

Another object of the present invention is to provide a waterless, multi-purpose product having the characteristic features described above which provides the desired hand and/or skin cleaning and sanitizing, while also enabling an individual to exercise hand muscles.

Another object of the present invention is to provide a waterless, multi-purpose product having the characteristic features described above which is also employable for removing dirt, grease, and other contamination from the skin surface, for effectively cleaning the skin surface while also providing the sanitizing and disinfecting results.

Other and more specific objects will in part be obvious and will in part appear hereinafter.

### DETAILED DISCLOSURE

By employing the present invention, all of the difficulties and drawbacks found in the prior art have been eliminated and a highly effective and easily employed, multi-purpose hand and/or skin cleaner, sanitizer, drug delivery, and exercise system is realized. In accordance with the present invention, a reusable support base or carrier is employed which is constructed for being easily held by the user in the person's hand. In addition, the support base or carrier is formed from soft, pliable, compressible and/or flexible material which is easily manipulated by the user in the person's hand or on the skin surface of other areas of the person's body. As a result, by repeatedly flexing one's fingers, the support base or carrier is easily compressed, formed, manipulated, and reformed into a plurality of alternate shapes and configurations, thereby enabling the user to employ a wide variety of muscles which control the fingers and hands of the user. In this way, finger exercising is easily and conveniently achieved.

Furthermore, in accordance with the present invention, the soft, flexible, pliable and/or compressible support base/carrier incorporates additives, therapeutic agents and/or physical characteristics for providing, enhancing, and/or improving a variety of desirable and previously unattainable results. In this regard, one or more additives and/or therapeutic agents selected from the group consisting of anti-microbial agents, sanitizing agents, medicinal compounds, drugs, anti-bacterial agents, antiseptic agents, anti-viral agents, fragrances, oils, herbs, moisturizing agents, softening agents, and the like are incorporated into the support base or carrier for enabling the user to benefit from the particular additives and the individual results obtainable from these agents. In this way, a multi-purpose, highly effective, and unique hand or skin cleaning, sanitizing, and drug delivery system is realized, in combination with a system which also improves and enhances the individual's ability to obtain exercising of the hand.

Although various materials and compositions can be employed for the support base or carrier of the present invention, it has been found that silicone-based materials are preferably employed for their inherent physical quality of being soft, pliable, and highly elastic. Furthermore, silicone-based materials remain pliable and continue to exhibit elastic qualities at temperature extremes of both hot and cold providing similar user utility in both summer and winter environments. Although silicone-based materials meeting these criteria are preferred for use as the support base/carrier of the present invention, other materials having these desirable physical characteristics may be employed with equal efficacy.

In addition, in selecting the preferred silicone-based material for the base/carrier of the present invention, or any equivalent material as a substitute therefor, the composition preferably comprises an adhesive or sticky physical characteristic as an inherent quality thereof. In this way, the repeated use of the carrier/base in the hand of the user by rolling, kneading, squeezing, and/or manipulating the material also causes any surface contaminants on the hand of the user to be automatically removed from the hand surface and integrated into the carrier/support base by virtue of the sticky physical characteristic. In this way, bacteria, viruses, surface contaminants, dirt, grease, etc., are all automatically removed from the surface of the hand of the user and absorbed into the support base/carrier in a water-free cleaning process. Once absorbed onto the support base/carrier, the anti-microbial agents, anti-bacterial agents, anti-septic agents, and/or sanitizing agents render the absorbed organisms harmless over time.

Furthermore, in the preferred embodiment of the present invention, various additives and/or agents are incorporated into the support base/carrier for further enhancing the performance of the multi-purpose hand cleaner, sanitizer, and drug delivery system of the present invention. In this regard, these additives and/or agents preferably comprise one or more selected from the group consisting of anti-microbial agents, anti-bacterial agents, antiseptic agents, anti-viral agents, sanitizing agents, medicinal compounds, drugs, fragrances, oils, herbs, moisturizing agents, softening agents, and the like for providing the user with specifically desired result-oriented enhancements. By incorporating anti-microbial agents, anti-bacterial agents, antiseptic agents, anti-viral agents, sanitizing agents, and the like, the multi-purpose system of the present invention is capable of neutralizing and/or eliminating any viruses, bacteria, germs, etc. which may be present on the hands of the user.

In addition, by incorporating medicinal compounds or drugs, such as aspirin, acetaminophen, benzocaine, and the like, the particular drugs are capable of being efficiently and effectively delivered to the user by transdermal transmission through the skin surface of the user into the bloodstream. In this way, these drugs or medicines are efficiently and effectively delivered directly into the bloodstream of the user in a simple, easily achieved manner. It has been found that these medicinal compounds or drugs are preferably incorporated into the support base/carrier in quantities ranging between about 5% to 50% by weight based upon the weight of the entire composition, with a quantity ranging between about 100% and 25% by weight based upon the weight of the entire composition being optimal.

By incorporating further agents in the support base/carrier of the present invention, such as fragrances, oils, herbs, moisturizing agents, coloring agents, texture-modifying agents, softening agents, exothermic or endothermic agents, and the like, additional desirable skin surface treatments are quickly and easily transmitted to the skin of the user in an effective and easily employed system. In this way, the skin surface of the user's hands, feet, arms, shoulders, etc. can be easily treated with a wide variety of various components and ingredients for imparting to the skin surface desirable agents or enhancements, which are otherwise difficult to achieve.

By forming the support base/carrier of the present invention from silicone-based materials, or equivalent materials, the multi-purpose, cleaning/sanitizing, drug delivery, and exercising system of the present invention is capable of being reused for extended periods of time. Typically, the support base/carrier can be repeatedly used until the efficacy of the ingredients incorporated therein has been fully spent. In this regard, if desired, a color altering compound can be incorporated into the support base/carrier for changing from a first color representing usability of the support base/carrier into an alternate color, representing that the components incorporated therein have been exhausted. In this way, the user is able to immediately know when the efficacy of the ingredients incorporated therein has been exhausted and a new support base/carrier system should be used.

Furthermore, as is evident from the foregoing detailed disclosure, the support base/carrier of the present invention operates in a waterless environment, thereby enabling the delivery of any desired ingredient as well as the cleaning of hands or skin surface by merely rubbing or manipulating the support base/carrier over the skin surface, devoid of any need for water. As a result, added versatility and ease of use of the present invention is realized.

Throughout the foregoing detailed disclosure, silicone is employed for referring to polysiloxanes which are generally inorganic-organic polymers formed by organic groups, such as methyl, ethyl, and phenyl, in combination with inorganic silicon-oxygen component wherein the organic site groups are attached to the silicon atoms. In this way, a wide variety of physical properties are capable of being achieved for assuring that the support base/carrier comprises the precisely desired physical characteristics.

Although a wide variety of alternate silicone formulations or compositions can be employed in forming the support base/carrier of the present invention, it has been found that the preferred silicone formulation is SG-135, manufactured by Shincor Silicones, Inc. This preferred silicone formulation comprises a general-purpose silicone rubber base which is typically used in a variety of alternate applications. In addition, although most prior art applications employing SG-135 silicones employ compositions mixed with other materials and post cured in a thermal process to yield a tough and resilient silicone rubber material, the formulations employed in the present invention are used in its “uncured” or “non-cured” state, wherein flow characteristics and adhesive-like properties are still exhibited. In this way, desirable physical attributes for use in the present invention are attained.

As fully discussed above, a wide variety of additives, therapeutic agents, medicinal compounds, and/or cosmetic compounds can be incorporated into the support base/carrier of the present invention in order to impart particular, desirable, characteristics and/or attributes to the delivery system of the present invention. In this regard, one such additive or therapeutic agent which has been found to be highly effective in combination with the support base/carrier of this invention is the anti-microbial agent known as AlphaSan, manufactured by Milliken & Co. By incorporating AlphaSan into the support base/carrier of the present invention, a highly effective delivery system is achieved for protecting individuals against various micro-organisms, including microbes, germs, bacteria, fungi, and algae.

AlphaSan is an anti-microbial additive which comprises a zirconium phosphate-based ceramic ion exchange resin containing silver. By incorporating an effective amount of AlphaSan into the support base/carrier of the present invention, bacteriostatic, fungistatic, and algistatic properties are imparted thereto. In this way, a support base/carrier of the present invention is capable of providing these desirable characteristics to the user employing the present invention.

Alternatively, similar anti-microbial properties may be achieved with use of a triclosan-based products such as Microban™ “Additive “B”, manufactured by the Microban Products Company of Huntersville, N.C. At in-use concentrations triclosan acts as a biocide, with multiple cytoplasmic and membrane targets. However, at lower concentrations, triclosan appears bacteriostatic and is seen to target bacteria mainly by inhibiting fatty acid synthesis. It has been found that the anti-microbial agents are preferably incorporated into the support base/carrier in quantities ranging between about 0.01% and 1% by weight based upon the weight of the entire composition.

In addition, as fully detailed above, a wide variety of other additives, therapeutic agents, medicinal compounds, and/or cosmetic compounds can be incorporated into the support base/carrier of the present invention to achieve a delivery system which is either broad-based or constructed for selective, specific therapies. Regardless of which component and/or combination of component are employed in the support base/carrier of the present invention, a unique delivery system is realized, which also provide desirable skin surface cleaning and hand exercise.

An alternate use of the delivery system of the present invention which has been found to provide a further enhanced results is the incorporation of medicinal compounds into the support base/carrier of the present invention and the formation of the support base/carrier as an ear plug. In this way, any desired medicines, drugs, and similar compounds are effectively delivered directly to the ear canal of a user for imparting these medicinal compounds to the bloodstream of the user through the ear cannel. In this way, drug delivery can be achieved more directly, as opposed to the transdermal transmission otherwise achieved from applying the drug incorporating support base/carrier of the present invention to a particular skin surface location.

### BEST MODE FOR CARRYING OUT THE INVENTION

In order to substantiate the versatility and substantial anti-microbial and anti-bacterial benefits provided by this particular embodiment of hand and/or skin cleanser/sanitizer of the present invention, the following examples are presented. In the following disclosure, the universal applicability of the present invention is detailed, along with the ability of the anti-microbial/anti-bacterial additives incorporated into the support base/carrier of the present invention to completely eradicate a wide variety of disease causing micro-organisms, bacteria, and the like. It is to be understood, however, that these examples are intended as a teaching of the best mode for carrying out this particular embodiment of the present invention and are not intended to limit, in any manner, the breadth of this discovery.

In order to prove the efficacy of this embodiment of the present invention and clearly establish the capability of this embodiment to completely eliminate disease causing micro-organisms, bacteria, and the like, the following tests were conducted. In the following tests, several batches of uncured silicones were prepared with each batch of silicone having different durometers. In this way, the effect, if any, of the durometer of the silicone would be determined.

In addition to employing silicone compositions having different durometers, two separate and distinct anti-microbial or anti-bacterial additives were also tested. In this regard, one anti-microbial/anti-bacterial additive comprised a triclosan-based composition identified as Microban Additive B. The second anti-microbial additive tested is identified as Microban Additive IB5, which is a silver based anti-microbial compound. Both of these additives are manufactured and sold by Microban Products Company of Huntersville N.C.

In order to determine the efficacy of the anti-microbial or antibacterial additives, the AATCC Test Method 100 is normally employed. This test method, developed by the American Association of Textile Chemists and Colorists, is employed to provide a quantitative procedure for the evaluation of the degree of anti-bacterial/anti-microbial activity on textile materials. In accordance with this test method, the surface of the samples being tested are inoculated with a known number of microbes and then the microbes are recovered to see if any growth or inhibition has occurred. However, the silicone samples employed in this invention are extremely sticky and tacky and, as a result, it was not possible to recover the microbes after the incubation period. As a result, a modified method was developed.

In accordance with the modified procedure, the silicone test samples were inoculated with the microbes and incubated for 24 hours. After the 24 hours, a sterile agar was poured over the samples. The sample was left to incubate for another 24 hours and then examined to see if any microbial growth had occurred. If the anti-microbial treated samples had killed all of the bacteria, the agar would remain clear. If the bacteria population has survived and/or grown, then the agar would show the growth. Although this method is not quantitative, since the number of microbes are not enumerated, a qualitative estimation or ranking of the samples is possible, thereby achieving an evaluation of the relative efficacy of the samples.

In evaluating the efficacy of the triclosan-based anti-microbial additive, the silicone samples were prepared with durometers of 25, 35, and 40. Each of the three different silicone samples were exposed to three different concentrations of the triclosan, namely 800 ppm, 1500 ppm, and 2500 ppm. In addition, untreated control samples were also prepared and tested.

The typical test procedure employed is in accordance with AATCC Test Method 90, wherein microbe bearing agar is poured on the samples and, after incubating, the samples are checked to see if the microbes in the agar have grown or have been killed. Following this procedure, the samples being tested were evaluated.

In determining the efficacy of the present invention in combination with the silver based anti-microbial additive, the modified AATCC Test Method 100, detailed above, was employed. In conducting this test, the silicone sample comprises only a durometer of 35. However, three separate and distinct concentrations of the anti-microbial additive were evaluated, namely concentrations of 7500 ppm, 10,000 ppm, and 20,000 ppm. In addition, an untreated control sample was also tested.

Once all test results were completed and all samples were evaluated, it was discovered that all concentrations in all samples of the triclosan-based antimicrobial additive performed well. Bacteria free zones around the samples were found for both Klebsiella Pneumonias and Staphylococcus Aureus. No variations were found in the test samples due to the concentration variations or for the different silicone durometers. All of the test samples work equally effectively, particularly when compared to the untreated control samples.

In the test results produced by employing the silicone-based anti-microbial additive, the concentration level of 800 ppm was found to be borderline effective. However, at concentrations of 10,000 ppm and 20,000 ppm, the anti-microbial additives were fully effective.

It will thus be seen that the objects set forth above, among those made apparent from the preceding description, are efficiently attained and, since certain changes may be made in the above product without departing from the scope of the invention, it is intended that all matter contained in the above description shall be interpreted as illustrative and not in a limiting sense.

It is also to be understood that the following claims are intended to cover all of the generic and specific features of the invention herein described, and all statements of the scope of the invention which, as a matter of language, might be said to fall there between.

Having described our invention, what we claim is new and desire to secure by Letters Patent is:

1. A hand and/or skin cleanser/sanitizer comprising:
A. a carrier formed from soft, flexible, pliable and/or compressible material which is easily manipulated into any desired shape; and
B. one or more additives incorporated into the carrier and comprising at least one selected from the group consisting of anti-microbial agents, sanitizing agent, antibacterial agents, antiseptic agents, and antiviral agents constructed for neutralizing any germs, bacteria, and/or microbes remove from the skin surface;
whereby a hand and/or skin cleanser/sanitizer is achieved which is capable of being easily used for cleaning and disinfecting hand surfaces and/or any skin surface by merely rubbing the carrier thereon.

2. The hand and/or skin cleanser/sanitizer defined in claim 1, wherein said carrier is further defined as comprising a silicone-based material having soft, pliable, and highly elastic physical properties.

3. The hand and/or skin cleanser/sanitizer defined in claim 2, wherein said carrier is still further defined as possessing an inherent adhesive or sticky physical quality for automatically removing surface contaminants from the hand or skin of the user whenever the carrier is applied thereto and/or rolled, kneaded, squeezed, or otherwise manipulated by the user, thereby enabling as the additive incorporated into the carrier to neutralize the removed contaminants.

4. The hand and/or skin cleanser/sanitizer defined in claim 1, wherein the additives incorporated into the carrier is further defined as comprising one or more are selected from the group consisting of fragrances, oils, herbs, moisturizing lotions, coloring agents, texture modifying agents, softening agents, exothermic agents and endothermic agents, whereby additional hand and/or skin treatments are effectively applied to the user.

5. The hand and/or skin cleanser/sanitizer defined in claim 1, wherein said carrier further comprises coloring altering compounds incorporated therein for effectively informing the user when the efficacy of the additives is exhausted and/or is in the process of being exhausted.

6. The hand and/or skin cleanser/sanitizer defined in claim 1, wherein said carrier is further defined as comprising a silicone and/or polysiloxane based material formed in a “non-cured” or “uncured” state wherein flow characteristics and adhesive-like properties are maintained.

7. The hand and/or skin cleanser/sanitizer defined in claim 1, wherein said additive is further defined as comprising one or more selected from the group consisting of medicinal compounds, drugs, and/or therapeutic agents.

8. The hand and/or skin cleanser/sanitizer defined in claim 7, wherein said additive comprises one or more selected from the group consisting of aspirin, acetaminophen, benzocaine, and pain relieving/reducing compounds.

9. The hand and/or skin cleanser/sanitizer defined in claim 8, wherein said additive is further defined as comprising between about 5% and 50% by weight based upon the weight of the entire composition.

10. The hand and/or skin cleanser/sanitizer defined in claim 1, wherein said additive comprises an anti-microbial additive consisting of zirconium phosphate-based ceramic ion exchange resins containing silver for providing bacteriostatic and algistatic properties to the carrier.

11. The hand and/or skin cleanser/sanitizer defined in claim 10, wherein said additive comprises between about 0.01% and 1.0% by weight based upon the weight of the entire composition.

12. The hand and/or skin cleanser/sanitizer defined in claim 11, wherein said additive comprises a concentration ranging between about 7500 ppm and 20,000 ppm.

13. The hand and/or skin cleanser/sanitizer defined in claim 1, wherein said additive comprises a triclosan-based compound.

14. The hand and/or skin cleanser/sanitizer defined in claim 13, wherein said additive comprises a concentration ranging between about 1000 ppm and 2500 ppm.

15. The hand and/or skin cleanser/sanitizer defined in claim 1, wherein the silicone-based material having a durometer ranging between about 25 and 40.
